(19) United States
(12) Patent Application Publication
(10) Pub. No.: US 2022/0008607 A1
(43) Pub. Date: Jan. 13, 2022

(54) Room Sterilization System

(71) Applicant: Jeffrey Paul Blaevoet, Novato, CA (US)

(72) Inventor: Jeffrey Paul Blaevoet, Novato, CA (US)

(21) Appl. No.: 17/370,813

(22) Filed: Jul. 8, 2021

Related U.S. Application Data
(60) Provisional application No. 63/049,389, filed on Jul. 8, 2020.

(51) Int. Cl. A61L 9/20; A61L 2/10; A61L 2/24; A61L 2/26

(52) U.S. Cl. CPC A61L 9/20; A61L 2/10; A61L 2/24; A61L 2/26; A61L 2202/14; A61L 2209/111; A61L 2202/25; A61L 2202/11; A61L 2209/12

(57) ABSTRACT

A room sterilization system generates ultraviolet germicidal irradiation (UVGI) to decontaminate a room whenever the room is free of occupants. To achieve this, the sterilization system has a light fixture, and an environmental sensor. The light fixture is a lamp used to produce UVGI for sterilizing the room, and has a housing, a sterilizing light source, and a microcontroller. The sterilizing light source is a UVGI emitter that is mounted within the housing and electrically connected to the microcontroller. The environmental sensor monitors the room to detect the presence of an occupant and then alerts the microcontroller to deactivate the sterilizing light source if the occupant is detected.

The current application claims a priority to the U.S. Provisional Patent application Ser. No. 63/049,389 filed on Jul. 8, 2020.

### FIELD OF THE INVENTION

The present invention relates generally to a cleaning system. More specifically to a system that uses radiation to decontaminate the air and objects within a given space.

### BACKGROUND OF THE INVENTION

Ultraviolet germicidal irradiation (UVGI) is a disinfection method that uses short-wavelength ultraviolet (or UV-C) light to kill or inactivate microorganisms. The method of employing UVGI for disinfection is commonly used in medical sanitations for sterilization.

UVGI destroys viruses such as SARS and Covid-19 but is also harmful to humans. UVGI can be built into HVAC systems by irradiating cooling coils, ducts, and filters to provide sterile air for operating rooms, health care facilities, offices, homes, and every kind of occupied space. This is a good method of dealing with aerosol air-borne viruses but does not address surfaces that the aerosols land on.

To treat surfaces such as desks, chairs, phones, keyboards, and others that people touch, it is clear that lighting systems are designed to illuminate these upward-facing surfaces. The present invention aims to overcome this by way of incorporating UVGI into regular lighting fixtures, allowing UVGI to be applied when the space is not occupied.

### BRIEF DESCRIPTION OF THE DRAWINGS

FIG. 1 is an illustration showing the present invention in the inactive state while an occupant is in the room.

FIG. 2 is an illustration showing the present invention in the active state while no occupant is in the room.

FIG. 3 is a schematic view of an embodiment of the present invention that is designed to function as a self-contained lighting and sterilization system.

FIG. 4 is a block diagram showing the connectivity between components of the present invention. In this view the present invention is used to retrofit an external lighting device.

FIG. 5 is a block diagram showing the connectivity between components of the present invention. In this view the present invention is a self-contained lighting and sterilization system.

FIG. 6 is a block diagram showing the present invention as a distributed network of light fixtures and environmental sensors that are managed by the remote server.

FIG. 7 is a flowchart illustrating the overall method for employing the present invention.

FIG. 8 is a flowchart illustrating a subprocess for activating the present invention while within a predefined time window.

FIG. 9 is a flowchart illustrating a subprocess for executing user commands through the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a heat recovery system, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data fingerprinting, role-based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer, etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touchscreen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

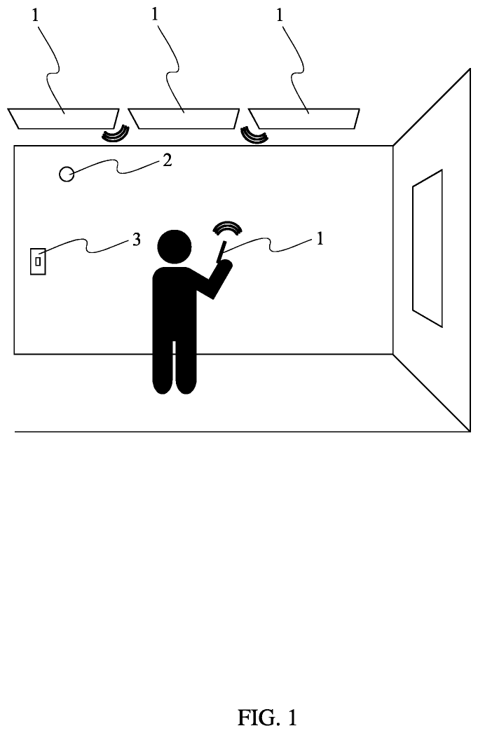

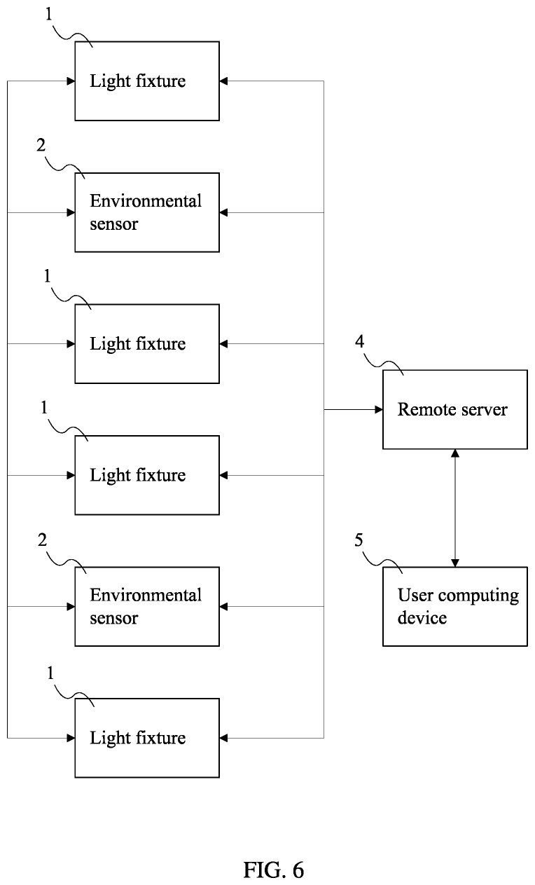

Referring to FIG. 1 through FIG. 9, the present invention, the room sterilization system, is an automated system designed to sterilize a room using ultraviolet germicidal irradiation (UVGI). By default, the present invention bathes the room in UVGI to disinfect the ambient air as well as any objects located within the room. Additionally, the present invention continuously monitors the room to determine if an occupant or living organism is within the room. If the occupant is detected, the present invention deactivates the UVGI generator until the occupant exits the room. Once the room is free of occupants, the UVGI generator is reactivated. Thus, preventing the present invention from exposing occupants to harmful UVGI.

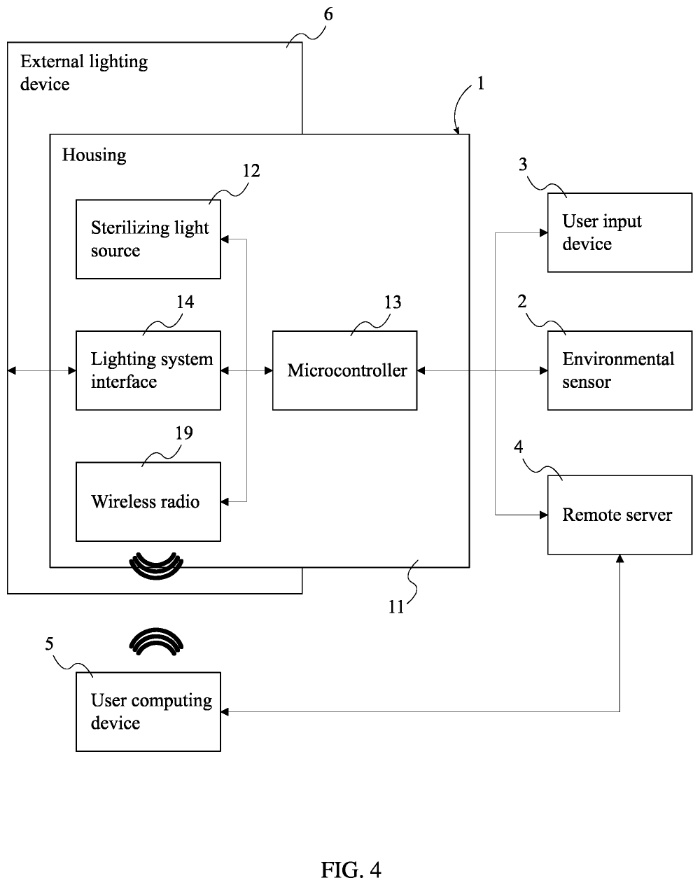

Referring to FIG. 4, to achieve the above-described functionality, the present invention comprises at least one light fixture 1 and at least one environmental sensor 2. Preferably, the present invention is used to retrofit an external lighting device. Thus, enabling a user to turn any room into a sterilized environment. The light fixture 1 is a self-contained UVGI generator that can be mounted onto, or integrated into, the external lighting device. The light fixture 1 comprises a housing 11, a sterilizing light source 12, and a microcontroller. The sterilizing light source 12 is a UVGI emitter that is mounted within the housing 11. Preferably, the housing 11 and the sterilizing light source 12 are configured to bathe a wide area of the room in UVGI. The microcontroller is an electronic control unit that governs the operation of the sterilizing light and the environmental sensor 2. Additionally, the microcontroller is mounted within the housing 11. The sterilizing light and the environmental sensor 2 are electronically connected to the microcontroller so that the microcontroller is able to send commands to the sterilizing light and interpret data gathered by the environmental sensor 2. The environmental sensor 2 is used to determine if a human is in the room. The environmental sensor 2 is at least one sensor selected from the group comprising cameras, acoustical sensors, biological material sensors, chemical sensors, accelerometers, motion sensors, infrared sensors, and ultrasonic sensors. In some embodiments the environmental sensor 2 is mounted onto the housing 11. Thus, the user is able to retrofit the external lighting device with the present invention. In alternative embodiments, the environmental sensor 2 is mounted offset from the housing 11. Accordingly, the environmental sensor 2 is advantageously placed to detect the presence of the occupant.

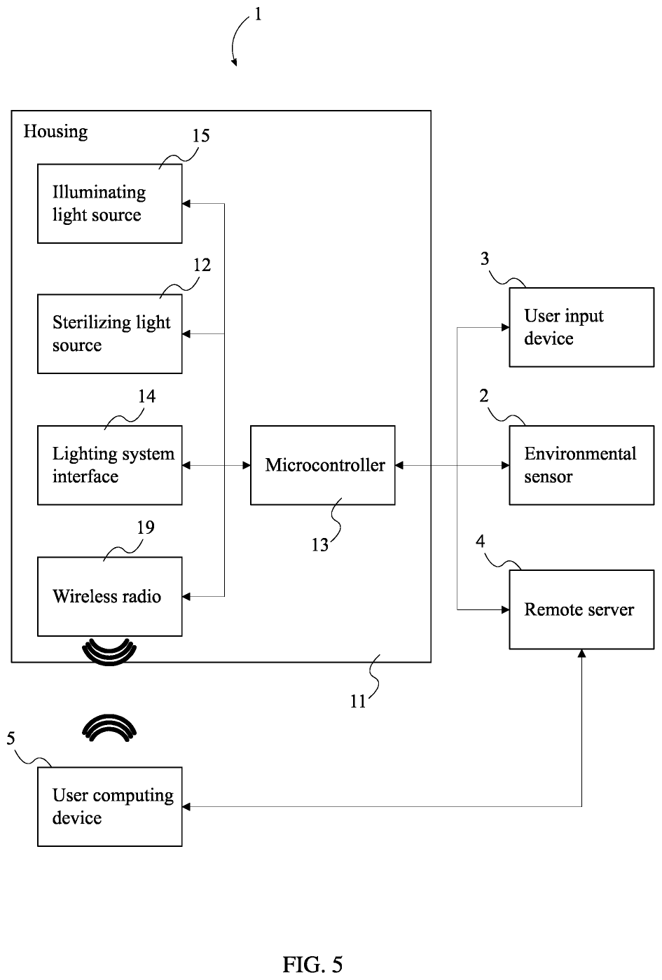

Referring to FIG. 5, the present invention is designed to function as a stand-alone sterilization device as well as a full-featured lighting device and sterilization system. To achieve this functionality, the present invention preferably comprises a lighting system interface 14. The lighting system interface 14 is an electrical coupling device that enables the present invention to be electrically connected to the external lighting device. Specifically, the lighting system interface 14 is electronically connected to the microcontroller and the external lighting device. Additionally, in this embodiment the housing 11 is mounted within the external lighting device. As a result, the microcontroller is able to control the operation of the external lighting device. Thereby, enabling the present invention to control both when the room is bathed in white light and when the room is bathed in UVGI.

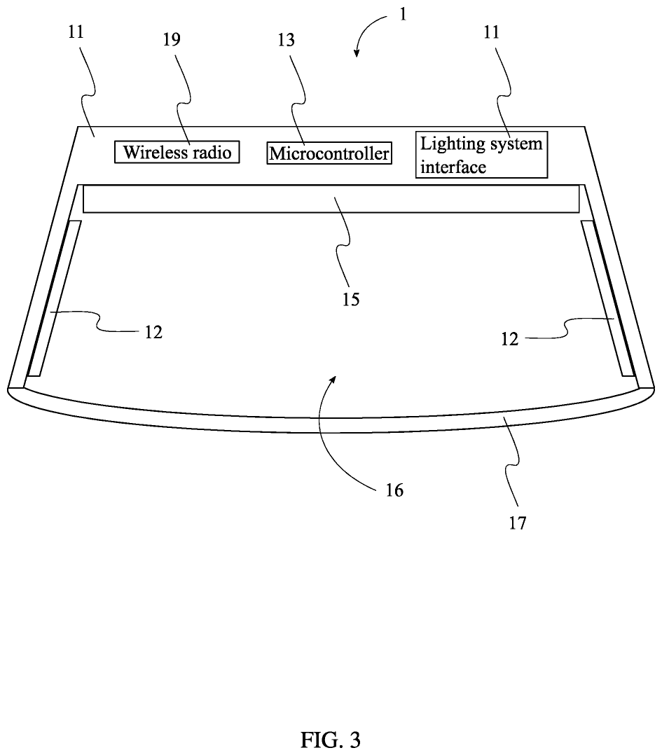

Referring to FIG. 3 and FIG. 4, in some embodiments, the present invention further comprises a wireless radio 19. The wireless radio 19 is electronically connected to the microcontroller so that the light fixture 1 is a able to be wirelessly connected to external systems. In some embodiments, the wireless radios 19 in a plurality of light fixtures 1 are communicably coupled to create an internet of things mesh network. In some embodiments, the wireless radio 19 acts as a wireless power transmitter and a WiFi access point.

Referring to FIG. 3 and FIG. 5, in alternative embodiments, the light fixture 1 further comprises an illuminating light source. The illuminating light source is a lamp that emits white light to illuminate the room. Additionally, the illuminating light source is mounted within the housing 11 and electronically connected to the microcontroller. Thus, the present invention is able to function as a full featured lighting device capable of producing both white light and UVGI. In these embodiments, the present invention is not designed to be a retrofit device, but rather functions as a standalone lamp capable of sterilizing the room. To further this functionality, the light fixture 1 further comprises an opening 16 and a light diffuser 17. The opening 16 traverses into the housing 11 and forms a receptacle within which the illuminating light source and the sterilizing light source 12 are mounted. Additionally, the illuminating light source and the sterilizing light are optically coupled to the opening 16 so that both white light and UVCI are able to exit the housing 11 and be projected into the room. The diffuser 17 is mounted over the opening 16 so that the white light produced by the illuminating light source is modified to create a pleasing lighting environment for the user. Additionally, the diffuser 17 is constructed from materials that do not hinder the UVGI from sterilizing the room.

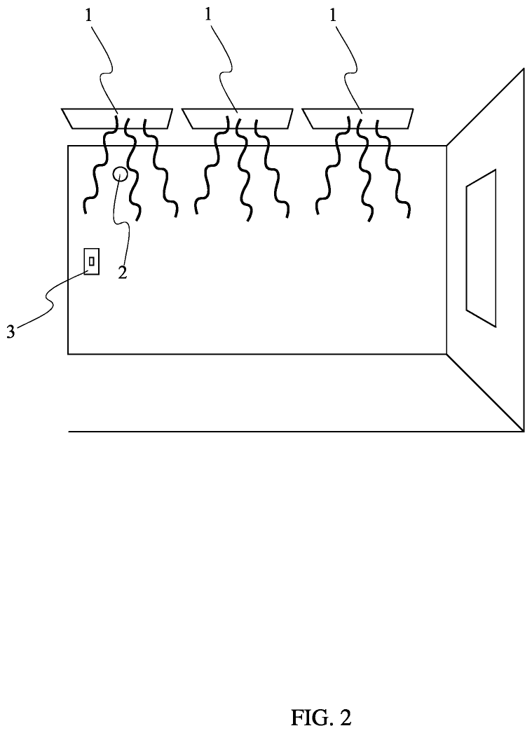

Referring to FIG. 1 and FIG. 2, the present invention further comprises at least one user input device 3. The user input device 3 is a physical control switch used to activate and deactivate the light fixture 1. The user input device 3 is any control device selected from the group comprising, switches, touch screen interfaces, and remote controllers. Additionally, the user input device 3 is positioned offset from the light fixture 1 and is communicably coupled to the microcontroller. Thus, the user input device 3 enables the user to override the microcontroller and activate or deactivate the sterilizing light source 12.

Referring to FIG. 3 through FIG. 6, in some embodiments, the present invention is designed to form a distributed network of UVGI emitters that are controlled and monitored remotely. To achieve this functionality, the present invention further comprises at least one remote server 4 and a user computing device 5. The term “remote server” is used herein to refer to a data processing system capable of running the background processes required to interpret the data from the environmental sensor 2 and communicating commands to the microcontroller. Additionally, the remote server 4 is responsible for relaying data between a variety of external systems and the light fixture 1. The user computing device 5 is an electronic device capable of communicating with the microcontroller and running the applications necessary to control the operation of the light fixture 1 and the environmental sensor 2. In some embodiments, a plurality of light fixtures 1 is distributed throughout an entire structure, or group of structures. The remote server 4 is used to monitor a plurality of environmental sensors 2 and the plurality of light fixtures 1 to control the lighting and sterilization operations required to illuminate and sterilize a plurality of rooms within the structures. The user computing device 5 provides an interface that displays all relevant information about the plurality of light fixtures 1 and the plurality of environmental sensors 2. Additionally, the user computing device 5 is communicably coupled to the remote server 4, such that the remote server 4 relays user input from the user computing device 5 to the microcontroller and the environmental sensor 2. As a result, the user is able to control one or more light fixtures 1 via the user computing device 5.

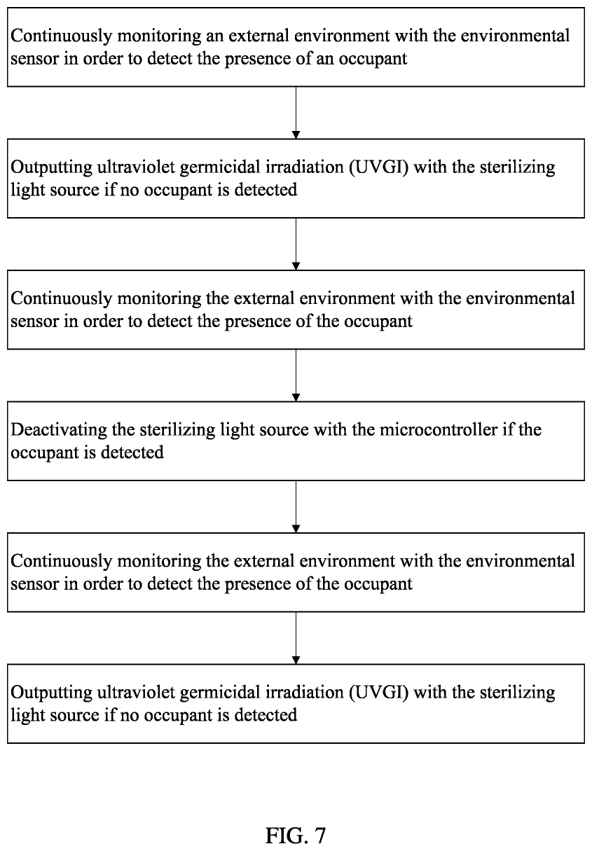

Referring to FIG. 5 and FIG. 7, the components enumerated above work in concert to form a system for sterilizing a room. Once the light fixture 1 is installed in the room, and electrically connected to a power source, the system begins by continuously monitoring the external environment within the room with the environmental sensor 2 in order to detect the presence of an occupant. Thus, the system captures a digital representation of the state within the room. The system then begins outputting UVGI with the sterilizing light source 12 if no occupant is detected. In some embodiments, the environmental sensor 2 is used to identify the presence of unwanted biological material within the room and activate the sterilizing light source 12 to decontaminate the room once no occupant is detected. The system then continues monitoring the external environment with the environmental sensor 2 in order to detect the presence of an occupant. The microprocessor flags the presence of the occupant as a change in state within the digital representation of the room. The microcontroller then issues commands to deactivate the sterilizing light source 12 with the microcontroller if an occupant is detected. This process is then repeated indefinitely until the system is deactivated by the user. As a result, the present invention provides a sterilization system that continuously decontaminates rooms without exposing occupants to harmful UVGI.

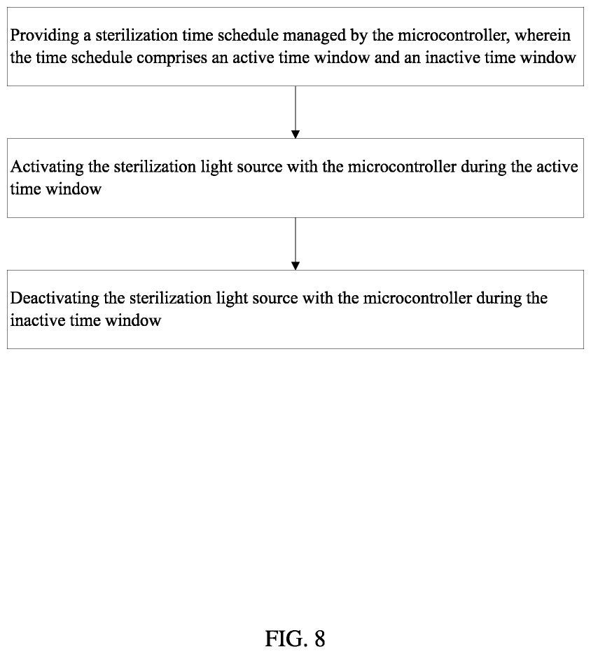

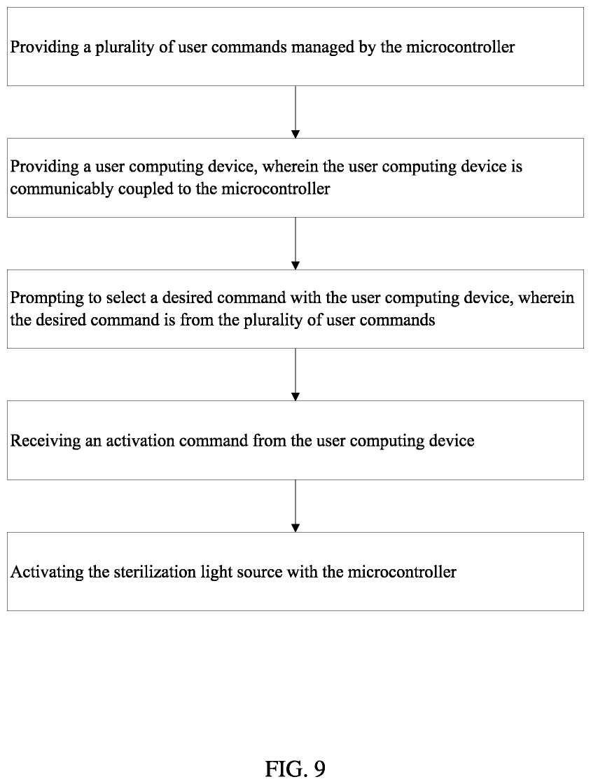

Referring to FIG. 5, FIG. 8, and FIG. 9, various additional method for controlling the sterilizing light source 12 exist such as commands issued from the user computing device 5, the remote server 4, or from any capably equipped electrical system. For example, embodiments of the present invention provide a sterilization time schedule managed by the microcontroller. The time schedule comprises an active time window and an inactive time window. Thus, the system is able to proceed activating the sterilization light source with the microcontroller during the active time window and deactivating the sterilization light source with the microcontroller during the inactive time window. The system provides a plurality of user commands managed by the microcontroller in order to enable further control of the sterilizing light source 12. The system prompts the user to select a desired command with the user computing device 5. The desired command is one of the plurality of user commands and enables the user to direct the light fixture 1 to perform any number of desired operations. For example, when the desired command received from the user device is an activation command, the system proceeds to activate the sterilization light source with the microcontroller.

Although the invention has been explained in relation to its preferred embodiment, it is to be understood that many other possible modifications and variations can be made without departing from the spirit and scope of the invention as hereinafter claimed.

What is claimed is:

1. A room sterilization system comprising:
at least one light fixture;
at least one environmental sensor;
the light fixture comprising a housing, a sterilizing light source, and a microcontroller;
the sterilizing light source being mounted within the housing;
the microcontroller being mounted within the housing; and
the sterilizing light source and the environmental sensor being electronically connected to the microcontroller.

2. The room sterilization system as claimed in claim 1 comprising:
the light fixture further comprising a lighting system interface;
the lighting system interface being electronically connected to the microcontroller and an external lighting device; and
the housing being mounted within the external lighting device.

3. The room sterilization system as claimed in claim 1 comprising:
the light fixture further comprising a wireless radio;
the wireless radio being electronically connected to the microcontroller and an external lighting device; and
the wireless radio being mounted within the housing.

4. The room sterilization system as claimed in claim 1 comprising:
the light fixture further comprising an illuminating light source;
the illuminating light source being mounted within the housing; and
the illuminating light source being electronically connected to the microcontroller.

5. The room sterilization system as claimed in claim 4 comprising:
the light fixture further comprising an opening and a light diffuser;
the opening traversing into the housing;
the illuminating light source and the sterilizing light source being optically coupled to the opening; and
the diffuser being mounted over the opening.

6. The room sterilization system as claimed in claim 1 comprising:
at least one user input device;
the user input device being positioned offset from the light fixture; and
the user input device being communicably coupled to the microcontroller.

7. The room sterilization system as claimed in claim 1 comprising:
at least one remote server; and
the remote server being communicably coupled to the microcontroller, wherein the remote server governs the operation of the sterilizing light source.

8. The room sterilization system as claimed in claim 7 comprising:
a user computing device; and
the user computing device being communicably coupled to the remote server, wherein the remote server relays user input from the user computing device to the microcontroller.

9. A system for sterilizing a room comprising:
at least one light fixture;
at least one environmental sensor;
the light fixture comprising a housing, a sterilizing light source, and a microcontroller;
the sterilizing light source and the environmental sensor being electronically connected to the microcontroller;
continuously monitoring an external environment with the environmental sensor in order to detect the presence of an occupant;
outputting ultraviolet germicidal irradiation (UVGI) with the sterilizing light source if no occupant is detected;
continuously monitoring the external environment with the environmental sensor in order to detect the presence of the occupant;
deactivating the sterilizing light source with the microcontroller if the occupant is detected;
continuously monitoring an external environment with the environmental sensor in order to detect the presence of an occupant; and
activating the sterilizing light source with the microcontroller if no occupant is detected.

10. The system for sterilizing a room as claimed in claim 9 comprising:
providing a sterilization time schedule managed by the microcontroller, wherein the time schedule comprises an active time window and an inactive time window;
activating the sterilization light source with the microcontroller during the active time window; and
deactivating the sterilization light source with the microcontroller during the inactive time window.

11. The system for sterilizing a room as claimed in claim 9 comprising:
providing a plurality of user commands managed by the microcontroller;
providing a user computing device, wherein the user computing device is communicably coupled to the microcontroller;
prompting to select a desired command with the user computing device, wherein the desired command is from the plurality of user commands;
receiving an activation command from the user computing device; and
activating the sterilization light source with the microcontroller.
